(19) United States
(12) Patent Application Publication
(10) Pub. No.: US 2026/0063610 A1
(43) Pub. Date: Mar. 5, 2026

(54) SMART WATER SENSING AND MANAGEMENT SYSTEM

(71) Applicant: Delta Faucet Company, Indianapolis, IN (US)

(72) Inventors: Robert W. Rodenbeck, Indianapolis, IN (US); Klaus Brodum, Ann Arbor, MI (US); Joshua Drew Wales, Indianapolis, IN (US); Stephen Stec, Dearborn, MI (US)

(21) Appl. No.: 19/310,279

(22) Filed: Aug. 26, 2025

Related U.S. Application Data
(60) Provisional application No. 63/687,762, filed on Aug. 27, 2024.

(51) Int. Cl. G01N 33/18; G01N 27/416

(52) U.S. Cl. CPC G01N 33/1826; G01N 27/4167; G01N 27/4168

(57) ABSTRACT

A smart water sensing and management system is configured to operate in a tiered arrangement based upon various combinations of time, temperature and/or oxidant reductant potential (ORP) at point of use (POU), point of treatment (POT) and/or point of entry (POE) for controlling an electrically operable valve to purge water. The system is also configured to look for (a) low ORP levels and/or (b) high conductivity levels combined with a rapid drop in ORP levels to provide an indication of water quality to a user.

### CROSS-REFERENCE TO RELATED APPLICATION

The present application claims priority to U.S. Provisional Patent Application Ser. No. 63/687,762, filed Aug. 27, 2024, the disclosure of which is expressly incorporated herein by reference.

### BACKGROUND AND SUMMARY OF THE DISCLOSURE

The present invention relates generally to a smart water sensing and management system and, more particularly, to such a system including a sensor array and tiered controller solution for monitoring water quality and sequencing purging of water.

Key parameters in operating a safe water supply in critical locations such as hospitals, hospice, and retirement homes include management of heavy metal and biological activity in the water considered a risk to the user.

Without intervention, water entering a facility at a point of entry (POE) will attain an age in its passage to a point of use (POU). Age is associated with increased levels of contaminants stemming from the water grid and provides an opportunity for bacterial growth. More particularly, changes resulting from heavy metals and biological activity are associated with changes in temperature and a reduced level of oxidant reductant potential (ORP) typically originating from four sources, here called contributors:
Heavy metal: corrosion of pipes accumulating leachates such as iron, copper, zinc, lead;
Organics;
Anaerobe bacterial flora; and
Aerobe bacterial flora.
   All of these contributors add to the level of contaminants realized at the POE. The reduced level of oxidant reductant potential (ORP) may directly correlate with a loss of residual disinfectant as the residual disinfectants are oxidants. Once the residual disinfectants are gone, bacterial growth can be an order of magnitude larger.

The contribution from contributors can be predicted based on the time the water has resided in the pipe system (i.e., the age of the water). The contribution from contributors can be followed by or indicated by the ORP, and can be quantified by the difference in ORP (long term) and temperature (short term) between the POE and the POU.

An oxidation-reduction potential (ORP) sensor may measure the ability of a solution to either gain or lose electrons, which indicates how oxidizing or reducing the solution is. An ORP sensor may measure the electrical potential (e.g., in millivolts) between a measuring electrode, which is usually made of platinum or gold, and a reference electrode. A positive ORP value indicates an oxidizing environment, such as when there is chlorine in water. A negative ORP value indicates a reducing environment, such as wastewater with organic matter.

Consumers do not have an effective real-time means to determine whether their water is safe from harmful microbes. One way a consumer would know if their water had high bacteria levels was by sending a sample to a lab and receiving a report several weeks later. Another way a consumer would know if their water had high bacteria levels was by receiving a city-issued boil water alert. Conditions that could drive high bacteria levels include water main breaks (introducing bacteria) or water stagnation resulting in consumption of chlorine/chloramines which keep water safe from bacteria.

According to an illustrative embodiment of the present disclosure, a smart water sensing and management system includes an electronic controller, a timing device (e.g., a clock) in electrical communication with the controller, and a conduit defining a passageway for water. A temperature sensor is configured to detect temperature of the water and is in electrical communication with the controller. An oxidation reduction potential (ORP) sensor is configured to detect the activity of oxidizers and reducers in the water. An electrically operable valve is in electrical communication with the controller and is fluidly coupled to the conduit to control water flow therethrough to thereby cause water purging. The water purging is dependent upon an output of the timing device, the detected temperature of the water, and/or the detected activity of oxidizers and reducers in the water.

According to another illustrative embodiment of the present disclosure, a smart water sensing and management system includes a controller, a conduit defining a passageway for water, and a sensor array operably coupled to the conduit. The sensor array includes an oxidation reduction potential (ORP) sensor configured to detect the activity of oxidizers and reducers in the water, and a conductivity sensor configured to detect total dissolved solids (TDS) in the water. Illustratively, (1) low ORP levels, and/or (2)(a) an increase in conductivity levels and (b) a decrease in ORP levels cause the controller to provide an indication to a human user.

According to yet another illustrative embodiment of the present disclosure, a smart water sensing and management system includes an electronic controller, a conduit defining a passageway for water, and a sensor array operably coupled to the conduit. The sensor array includes an oxidation reduction potential (ORP) sensor in electrical communication with the controller and detecting the activity of oxidizers and reducers on the water. Low ORP levels cause the controller to purge the water from the conduit or provide an indication to a human user.

Additional features and advantages of the present invention will become apparent of those skilled in the art upon consideration of the following detailed description of the illustrative embodiment exemplifying the best mode of carrying out the invention as presently perceived.

### BRIEF DESCRIPTION OF THE DRAWINGS

The detailed description of the drawings particularly refers to the accompanying figures in which:

FIG. 1 is a block diagram of an illustrative smart water sensing and management system; and

FIG. 2 is a diagrammatic view of an illustrative sensor array of the smart water sensing and management system of FIG. 1.

### DETAILED DESCRIPTION OF THE DRAWINGS

For the purposes of promoting and understanding the principles of the present disclosure, reference will now be made to the embodiments illustrated in the drawings, which are described herein. The embodiments disclosed herein are not intended to be exhaustive or to limit the invention to the precise form disclosed. Rather, the embodiments are chosen and described so that others skilled in the art may utilize their teachings. Therefore, no limitation of the scope of the claimed invention is thereby intended. The present invention includes any alterations and further modifications of the illustrated devices and described methods and further applications of principles in the invention which would normally occur to one skilled in the art to which the invention relates.

The invention relates generally to a smart water sensing and management system and, more particularly, to such a system including a tiered solution for sequencing purges from point of use (POU) devices. The invention further relates generally to a smart water sensing and management system including a sensor array in wireless communication with a controller for monitoring water quality in residential water lines.

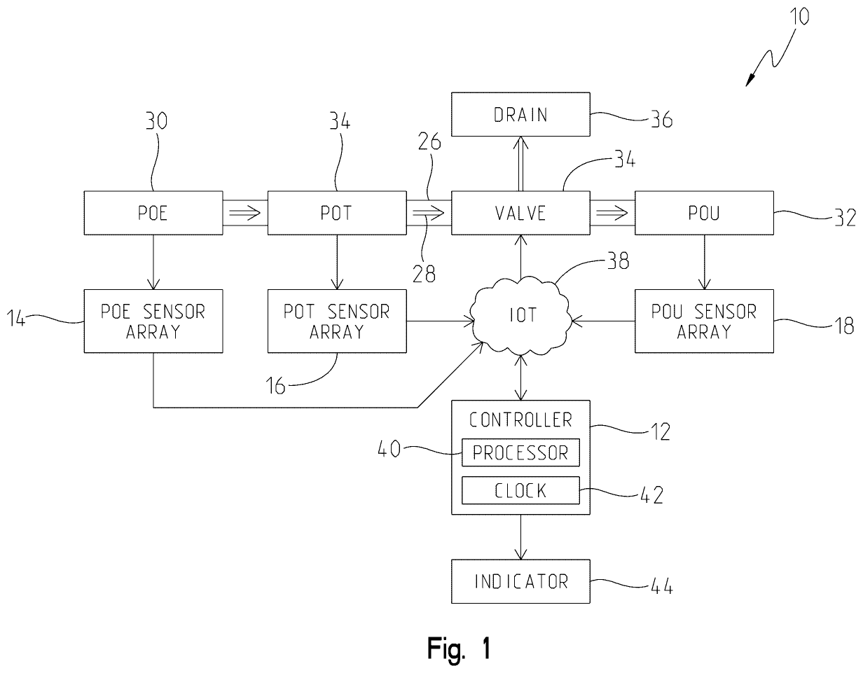

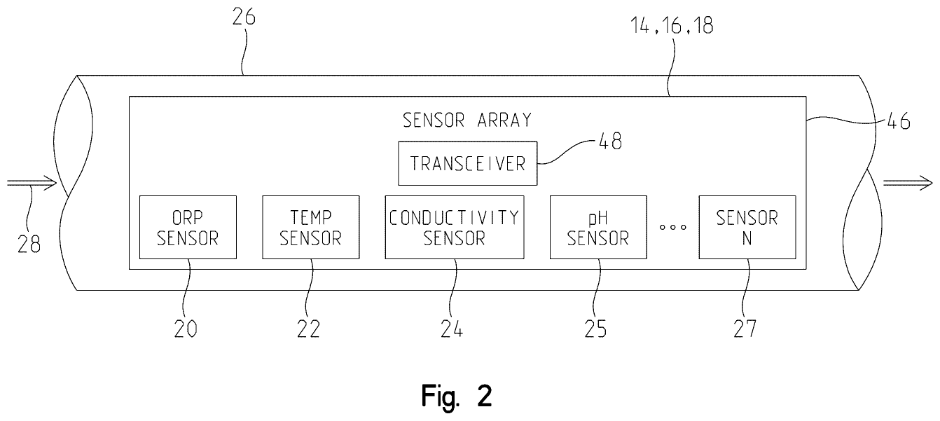

With reference to FIG. 1, the illustrative smart water sensing and management system 10 includes a controller 12 in communication with a plurality of water sensor arrays 14, 16 and 18. As shown in FIG. 2, each sensor array 14, 16 and 18 may comprise an oxidation reduction potential (ORP) sensor 20, a temperature sensor 22, a conductivity sensor 24, a pH sensor 25 and/or N number of additional sensors 27 to measure parameters of water within a conduit 26. Sensors 27 may include heavy metal sensors, multiplexed bioelectronic sensors, carbon nanofiber sensors, biosensors, turbidity sensors and/or dissolved oxygen sensors, for example. The conduit 26 illustratively defines a passageway 28 for the flow of water between a point of entry (POE) 30 and a point of use (POU) 32. An electrically operable valve 34 (e.g., a solenoid valve) is in communication with the controller 12 and may discharge water flow from the conduit 26 to a drain 36. In other words, the electrically operable valve 34 may purge water from the conduit 26 to the drain 36 as further detailed herein.

A point of treatment (POT) 37 is fluidly disposed between the POE 30 and the valve 34. POT 37 includes a water treatment device 39, such as an ozone generator, for example.

The ORP sensor 20 may be of conventional design for measuring the activity of oxidizers and reducers in the water within the conduit 26 and providing a signal indicative thereof to the controller 12. The temperature sensor 22 may also be of conventional design for measuring temperature of water within the conduit 26 and providing a signal indicative thereof to the controller 12. The conductivity sensor 24 may be of conventional design for measuring total dissolved solids (TDS) within the water in the conduit 26 and providing a signal indicative thereof to the controller 12. The sensors 14, 16 and 18 and/or the electrically operable valve 34 may be in wireless communication with the controller 12 via the cloud 38 (e.g., Internet of Things (IOT)).

The controller 12 may include a processor 40 (e.g., a central processing unit (CPU)) in electrical communication with an electronic timing device (e.g., a clock) 42. It is to be understood that the electronic timing device 42 does not necessarily display any particular time-of-day or time period as a clock might, however. An indicator 44 is in electrical communication with the controller 12. The indicator 44 may be a visual display as part of a user interface.

The smart water sensing and management system 10 illustratively provides a tiered Internet of Things (IoT) solution including a plurality of operating tiers or modes, with each tier relying on a limited purge enabled by electrically operable valves (e.g., solenoids) remotely controlled at a point of use (POU) device such as electronic faucet(s), electronic shower(s) and/or other remotely controlled solenoid-based units. The purge is illustratively determined by the following tiers:
Tier 1—time;
Tier 2—time and Oxidant Reduction Potential (ORP) at point of use (POU);
Tier 3—time and ORP at POU and point of entry (POE); and
Tier 4—time and ORP at POU, point of treatment (POT) and point of entry (POE).

Various algorithms allow the system to operate a Tier 1 solution based on time without additional feedback, and Tier 2-4 solutions including feedback from time, temperature and/or ORP.

The illustrative smart water management system of the present disclosure is set-up with POU, POT and POE devices connected via wireless protocol to interface enabling electrically operable valve (e.g., solenoid) actuation and communication of sensing and valve status in real time, according to Tier level sophistication.

One purpose of the purge is to reduce the age of the water. Water of age is purged and youthful water is reestablished. For example, the purge time is based on time, feedback, and device-specific parameters weighting different aspects of age-based contamination.

Sequenced purges from POU units are initiated from a controller following a schedule based on time and/or sensing of temperature and ORP as dictated by algorithms for concentration and ORP as compared to an operating window for temperature and ORP.

The efficacy of the purge can be established for Tier 2-4 systems and made a basis for machine learning and self-optimization.

The illustrative water safety sensing and management system 10 may include at least one sensor array 14, 16, 18 of the type shown in FIG. 2. Each sensor array 14, 16, 18 illustratively includes a support 46, such as a printed circuit board (PCB) supporting the ORP sensor 20, the temperature sensor 22 and/or the conductivity sensor 24. A transceiver 48 may also be supported on the support 46 and is configured to transmit and/or receive signals to/from the controller 12. Other sensor arrays may be substituted therefor, such as the multi-functional water quality sensor detailed in U.S. Patent Application Publication No. 2019/0025273 to Brondum et al., the disclosure of which is expressly incorporated herein by reference.

The illustrative water safety sensing and management system 10 of the present disclosure is configured to detect the quality of water within residential water lines. More particularly, through the use of low cost oxidation reduction potential (ORP) and conductivity (e.g., total dissolved solids (TDS)) sensors embedded in the water lines, the system may monitor the safety of water in real-time. In a first illustrative embodiment, the system looks for high water conductivity levels combined with a rapid change in ORP levels. In a second illustrative embodiment, the system monitors the absolute ORP level looking for conditions where the ORP level (e.g., the level of activity of oxidizers and reducers in the water) is low, indicating a consumption of chlorine/chloramines by organic matter.

According to the illustrative smart water sensing and management system 10, a sensor array (e.g., lab-on-a-chip) device is introduced in the water lines of a building. The illustrative sensor array comprises both a conductivity (e.g., total dissolved solids (TDS)) sensor and an oxidation reduction potential (ORP) sensor. The sensor array is connected (e.g., wirelessly) to a controller (e.g., a microprocessor device) which periodically measures both the conductivity and ORP based on signals from the conductivity sensor and the ORP sensor (e.g., at a sample rate of every 2-4 hours). The samples create a baseline of both conductivity and ORP levels within a building. After a baseline has been established, the microprocessor looks for (a) low ORP levels or (b) a notable increase in conductivity levels combined with a rapid drop in ORP levels. ORP levels below a certain threshold value indicate conditions where bacteria can grow, while high conductivities combined with a rapid change in ORP levels indicate undesirable bacteria levels, meaning that the water may be unsafe to drink. Although bacteria levels are not detected by a TDS meter, such as conductivity sensor 24, since bacteria are organic (electrically nonconductive), in some instances the bacteria interact with the water in such way to change total dissolved solids (TDS).

In an illustrative embodiment, drain 36 may be in the form of a smart (electronic) faucet that can be automatically activated or opened by controller 12 to purge the water in the lines. In one illustrative embodiment, there are more such smart faucets/drains than sensors. Each smart faucet may be at the end of a branch of plumbing, and thus, it is possible for controller 12 to purge each branch of plumbing individually.

Although the invention has been described in detail with reference to certain preferred embodiments, variations and modifications exist within the spirit and scope of the invention as described and defined in the following claims.

1. A smart water sensing and management system comprising:
an electronic controller;
a timing device in electrical communication with the controller;
a conduit defining a passageway for water;
a temperature sensor configured to detect temperature of the water and in electrical communication with the controller;
an oxidation reduction potential (ORP) sensor configured to detect the activity of oxidizers and reducers in the water; and
an electrically operable valve in electrical communication with the controller and fluidly coupled to the conduit to control water flow therethrough to thereby cause water purging, the water purging being dependent upon:
an output of the timing device;
the detected temperature of the water; and/or
the detected activity of oxidizers and reducers in the water.

2. The smart water sensing and management system of claim 1, wherein the conduit is positioned intermediate a point of entry (POE) of the water and a point of use (POU) of the water.

3. The smart water sensing and management system of claim 2, further comprising a treatment device positioned at a point of treatment (POT) intermediate the point of entry (POE) and the point of use (POU).

4. The smart water sensing and management system of claim 3, wherein the treatment device comprises an ozone generator.

5. The smart water sensing and management system of claim 1, wherein a sensor array is defined by the temperature sensor and the oxidation reduction potential (ORP) sensor.

6. The smart water sensing and management system of claim 5, wherein the sensor array further includes a conductivity sensor.

7. The smart water sensing and management system of claim 5, wherein the sensor array includes a circuit board received within the conduit.

8. A smart water sensing and management system comprising:
an electronic controller;
a conduit defining a passageway for water; and
a sensor array operably coupled to the conduit, the sensor array including:
an oxidation reduction potential (ORP) sensor configured to detect the activity of oxidizers and reducers in the water;
a conductivity sensor configured to detect total dissolved solids (TDS) in the water; and
wherein (1) low ORP levels, and/or (2)(a) an increase in conductivity levels and (b) a decrease in ORP levels cause the controller to provide an indication to a human user.

9. The smart water sensing and management system of claim 8, further comprising a timing device in electrical communication with the controller.

10. The smart water sensing and management system of claim 9, further comprising an electrically operable valve in electrical communication with the controller and fluidly coupled to the conduit to control water flow therethrough to thereby purge water from the conduit.

11. The smart water sensing and management system of claim 8, further comprising a temperature sensor configured to detect temperature of the water and in electrical communication with the controller.

12. The smart water sensing and management system of claim 8, wherein the conduit is positioned intermediate a point of entry (POE) and a point of use (POU).

13. The smart water sensing and management system of claim 12, further comprising a treatment device positioned at a point of treatment (POT) intermediate the point of entry (POE) and the point of use (POU).

14. The smart water sensing and management system of claim 8, wherein the sensor array includes a circuit board received within the conduit.

15. A smart water sensing and management system comprising:
an electronic controller;
a conduit defining a passageway for water; and
a sensor array operably coupled to the conduit, the sensor array including an oxidation reduction potential (ORP) sensor in electrical communication with the controller and configured to detect the activity of oxidizers and reducers on the water, wherein low ORP levels cause the controller to purge the water from the conduit or provide an indication to a human user.

16. The smart water sensing and management system of claim 15, wherein the sensor array includes a conductivity sensor in electrical communication with the controller, wherein the controller is configured to purge the water from the conduit or provide an indication to a human user dependent upon an output of the conductivity sensor.

17. The smart water sensing and management system of claim 15, further comprising an electrically operable valve in electrical communication with the controller and fluidly coupled to the conduit to control water flow therethrough to thereby purge the water from the conduit.

18. The smart water sensing and management system of claim 15, wherein the sensor array includes a temperature sensor in electrical communication with the controller and configured to detect temperature of the water, wherein the controller is configured to purge the water from the conduit or provide an indication to a human user dependent upon the detected temperature of the water.

19. The smart water sensing and management system of claim 15, wherein the conduit is positioned intermediate a point of entry (POE) and a point of use (POU), the system further comprising a treatment device positioned at a point of treatment (POT) intermediate the point of entry (POE) and the point of use (POU).

20. The smart water sensing and management system of claim 15, wherein the sensor array includes a pH sensor in electrical communication with the controller, wherein the controller is configured to purge the water from the conduit or provide an indication to a human user dependent upon an output of the pH sensor.
